(19) United States
(12) Patent Application Publication
(10) Pub. No.: US 2011/0118243 A1
(43) Pub. Date: May 19, 2011

(54) ANTI-INFLAMMATORY DRUG DELIVERY SYSTEM

(76) Inventor: Lynn Chambers, Grandbury, TX (US)

(21) Appl. No.: 12/933,632

(22) PCT Filed: Mar. 19, 2009
(86) PCT No.: PCT/US2009/037677

§ 371 Date: Dec. 21, 2010

Related U.S. Application Data
(60) Provisional application No. 61/038,326, filed on Mar. 20, 2008.

(51) Int. Cl. A61K 31/4152; A61K 31/196; A61K 31/407; A61K 31/192; A61K 31/381; A61K 31/5415; A61K 31/405; A61K 31/365; A61K 31/415; A61K 31/40; A61P 29/00; A61P 19/02

(52) U.S. Cl. 514/226.5; 514/404; 514/567; 514/411; 514/413; 514/570; 514/569; 514/448; 514/420; 514/473; 514/406; 514/423

(57) ABSTRACT

The invention provides dosage forms for the delivery of one or more non-steroidal anti-inflammatory drugs to equines. The dosage forms are palatable, desirable, and easy to administer with little or no mess or waste. The dosage form can be a cookie that includes one or more grain products, one or more protein products, one or more flavoring agents, or a combination thereof. The dosage form also includes a therapeutically effective amount of one or more non-steroidal anti-inflammatory drugs, for example, for the treatment of joint deterioration, swelling and inflammation, founder, fever, laminitis, or a combination thereof. The drug can be phenylbutazone or firocoxib and the dosage unit can be conveniently hand-fed to the horse.

### RELATED APPLICATIONS

This application claims priority under 35 U.S.C. §119(e) to U.S. Provisional Patent Application No. 61/038,326, filed Mar. 20, 2008, which is incorporated herein by reference in its entirety.

### BACKGROUND OF THE INVENTION

Non-steroidal anti-inflammatory drugs (NSAIDs) are among the most widely used drugs, due to their effective therapeutic properties as anti-inflammatories, analgesics, anti-pyretics, and anti-thrombolics. Furthermore, NSAIDs are used to treat a variety of clinical conditions manifesting such symptoms as pain, inflammation, fever, and to treat and prevent atherosclerosis.

Phenylbutazone, for example, is one of the most popular and useful nonsteroidal anti-inflammatory veterinary pharmaceuticals. Phenylbutazone is often the drug of choice for equine treatments when an illness or injury necessitates the use of a painkiller or anti-inflammatory medication. Phenylbutazone treats joint deterioration, swelling and inflammation from injuries, founder, fevers, and various other pains experienced by horses.

Phenylbutazone has been used to treat horses for more than 30 years and despite this long term acceptance of use, there are still many problems in effectively administering the drug. Phenylbutazone is most often administered orally and its bitter taste oftentimes causes the horse to reject the oral drug formulation, resulting in inconsistent dosages and difficulty in maintaining a prescribed treatment plan. Phenylbutazone is available to horse owners and veterinarians in one gram tablets for oral administration. Horses often will not willingly eat the tablets and the owner or veterinarian typically requires the aid of a handler in order to restrain the horse and/or to take other effective measures to orally administer the tablet.

Horse owners and veterinarians have developed some alternative means for the oral delivery of phenylbutazone to horses. In simple cases the tablets are crushed and mixed with the horse's food. This method is problematic because the crushed tablets typically do not adhere to the horse's food. Formulations in the form of a powder or granules readily sift to the bottom of a feeding trough as the horse consumes its food. The amount of sifting varies with each administration and results in inconsistent dosages or diet problems due to the addition of feed to administer the remaining medication.

Some horses reject the grain and drug mixture altogether, requiring the additional step of mixing the crushed tablet with syrup or molasses before adding a typically bitter NSAID to the horse's feed. This method is problematic because the mixing process can leave portions of the drug in the mixing container, feed trough, or dish, and the drug is typically insoluble in syrup or molasses, making it difficult or impossible to obtain a homogeneous mixture.

In other cases, the crushed tablets are mixed with water or a syrup to form a slurry for oral administration with a syringe. This method of delivery often requires the person administering the dosage to reach into the horse's mouth and exert pressure at certain points as inducement for the horse to open its mouth for the direct delivery of the drug to the horse's throat by syringe. This activity is unpleasant for the horse and for the person, and can result in injury if the person administering the drug is bitten, pawed, or stepped on by a frightened or stubborn horse. Because such a slurry is typically still bitter, a horse will continue to reject the slurry with efforts that increase in intensity over time. Ultimately, it becomes difficult or impossible to maintain a three times a day treatment regimen for delivery of NSAIDs. Horses often attempt to spit out such slurries after it is delivered.

While the administration of phenylbutazone and other NSAIDs in these forms is an inconvenience to horse owners and veterinarians, it is necessary for maintaining the health of the horse. In order to control or relieve inflammation and pain, a proper and effective dosage of an NSAID must be administered to the horse, repeatedly, over a particular time frame. In addition, the NSAID that is administered should be in the form that can undergo rapid intestinal absorption. Veterinarians typically prescribe a dosage to be administered three times a day because it takes approximately 3 to 5 hours for NSAIDS to achieve an effective blood concentration level. However, due to the problems with oral administration of tablets and similar forms, horse owners and even veterinarians often settle for a single daily administration, but using double or triple the dosage, which significantly reduces the efficacy of the NSAID.

Accordingly, what is needed is an NSAID dosage formulation that is acceptable and/or desirable to horses. What is also needed is an efficient, convenient, safe, and inexpensive method to deliver NSAIDs to horses.

### SUMMARY OF INVENTION

The present invention provides dosage forms for the delivery of one or more non-steroidal anti-inflammatory drugs to equines. The dosage forms are palatable, desirable, and easy to administer with little or no mess or waste, such as the loss of the drug to be delivered to the equine. The dosage form can be a cookie that includes one or more grain products, one or more protein products, one or more flavoring agents, or a combination thereof. The dosage form also includes a therapeutically effective amount of one or more non-steroidal anti-inflammatory drugs, for example, for the treatment of joint deterioration, swelling or inflammation, founder, fever, laminitis, or a combination thereof.

It has been surprisingly discovered that a unit dosage form that includes one or more non-steroidal anti-inflammatory drugs can be conveniently prepared by incorporating one or more non-steroidal anti-inflammatory drugs into a cookie that is specially designed for the taste buds of equines. Equines, such as horses, find the cookies to be delicious (i.e., desirable) and the handlers find the cookies to be a convenient and easy drug delivery system.

Each cookie can be made with a specific amount of one or more non-steroidal anti-inflammatory drug, along with any of a variety of desirable and palatable ingredients, such as grain products, protein products, flavoring agents, vitamins, minerals, preservatives, and/or amino acids. As a result, the cookie can be designed to be palatable, healthy, and have a long shelf life. The cookies can be packaged for veterinary and consumer use. When the drug is phenylbutazone, the cookie is referred to as a “bute cookie.”

Administering a cookie that is desirable to equines and that includes an NSAID offers several advantages over the traditional method of sprinkling NSAIDS, such as phenylbutazone, over the feed. First, the horse will quickly consume the cookie and thereby insure the desired intake of the NSAID in a timely manner. Second, there is no wasted NSAID. For example, if a phenylbutazone powder is sprinkled over the feed, a considerable amount of phenylbutazone will not land on the feed or stick to the feed, and will be lost on the bottom of the feeding trough. Further, some of the phenylbutazone powder that coats the feed may come off of the feed while the horse is eating the feed. Third, horses are significantly more willing to eat the desired cookie compared to the feed coated with the unpleasant tasting phenylbutazone.

Accordingly, the present invention provides a unit dosage form for the delivery of one or more non-steroidal anti-inflammatory drugs to equines comprising: a cookie comprising one or more grain products, one or more protein products, one or more flavoring agents, or a combination thereof; and a therapeutically effective amount of one or more non-steroidal anti-inflammatory drugs for the treatment of an equine ailment, wherein the ailment is joint deterioration, swelling and inflammation, founder, fever, laminitis, or a combination thereof.

In one embodiment, the one or more non-steroidal anti-inflammatory drugs include diclofenac, etodolac, ketorolac, bromfenac, ibuprofen, fenoprofen, fluriboprofen, ketoprofen, naproxen, suprofen, meclofenamate, mefenamic acid, piroxicam, meloxicam, indomethacin, sulindac, phenylbutazone, firocoxib, oxyphenbutazonetolmetin, celecoxib, or a pharmaceutically acceptable salt, metabolite, or prodrug thereof, or a combination thereof. Preferably, the one or more non-steroidal anti-inflammatory drugs is phenylbutazone or firocoxib. In one embodiment, the one or more non-steroidal anti-inflammatory drugs are present in the cookie in effective amounts so as to be palatable to horses.

In one embodiment, the one or more grain products include amaranth, barley, bran, buckwheat, corn, flax, millet, oats, rice, sorghum, triticale, rice, wheat, or a combination thereof. Preferably, the one or more grain products include bran, oats, or a combination thereof. In one embodiment, the one or more protein products include albumin, alfalfa, cheese, egg, milk, peanut, soy, whey, or a combination thereof. Preferably, the one or more protein products include alfalfa, whey, or a combination thereof. In one embodiment, the one or more flavoring agents include anise, apple fiber, banana, carrot, cherry, citric acid, cinnamon, fenugreek, molasses, orange, salt, raspberry, strawberry, vanillin, naturally expressed citrus or spice oils, or a combination thereof. Preferably, the one or more flavoring agents include anise, apple fiber, citric acid, fenugreek, molasses, or a combination thereof.

In one embodiment, the one or more non-steroidal anti-inflammatory drugs are present in an amount ranging about 2 percent by weight to about 6 percent by weight of the unit dosage form.

In one embodiment, the cookie ingredients in combination with the phenylbutazone is effective to accelerate blood absorption of phenylbutazone in equine blood subsequent to oral consumption of the unit dosage form, wherein acceleration of blood absorption is determined with respect to administration of phenylbutazone alone, for example, in a tablet form.

The present invention provides a method of administering one or more non-steroidal anti-inflammatory drugs to an equine comprising administering a cookie to an equine, wherein the cookie includes one or more grain products, one or more protein products, one or more flavoring agents, or a combination thereof; and a therapeutically effective amount of one or more non-steroidal anti-inflammatory drugs. The drugs can be for the treatment of an equine ailment, such as joint deterioration, swelling or inflammation, founder, fever, laminitis, or a combination thereof.

The present invention also provides a unit dosage form for the delivery of an NSAID, such as phenylbutazone or firocoxib, to a horse. The unit dosage for can include a cookie that includes one or more grain products, one or more protein products, one or more flavoring agents, or a combination thereof; and a therapeutically effective amount of the NSAID for the treatment of an equine ailment. The ailment can be joint deterioration, swelling or inflammation, founder, fever, laminitis, or a combination thereof, wherein the NSAID is present in an amount ranging about 2 percent by weight to about 6 percent by weight of the unit dosage form.

The present invention also provides a unit dosage form for the delivery of one or more non-steroidal anti-inflammatory drugs to a horse consisting essentially of a cookie as described herein. The present invention provides a method of administering one or more non-steroidal anti-inflammatory drugs to a horse consisting essentially of obtaining a unit dosage form consisting essentially of a cookie as described herein, and administering the cookie to a horse.

The present invention further provides a unit dosage form for the delivery of phenylbutazone to a horse consisting essentially of a cookie as described herein, wherein the NSAID, such as phenylbutazone or firocoxib, is present in an amount ranging from about 2 percent by weight to about 6 percent by weight of the unit dosage form.

The present invention provides a method of administering an NSAID, such as phenylbutazone, to an equine. A unit dosage form, for example, as a cookie as described herein, can be provided to an equine in need of an NSAID, by feeding the cookie to the equine. The cookie can include a therapeutically effective amount of an NSAID for the treatment of an equine ailment; the NSAID can be dispersed substantially homogeneously in the unit dosage form in an amount ranging from about 2 percent by weight to about 6 percent by weight of the unit dosage form. Equines such as domestic horses readily consume treats such as cookies, thereby effectively completing the administration.

In one embodiment, the step of obtaining a unit dosage form includes obtaining a unit dosage form wherein the cookie that includes the NSAID has ingredients present in an amount effective for accelerating blood absorption of the NSAID in equine blood subsequent to oral consumption of the unit dosage form, wherein acceleration of blood absorption is determined with respect to administration of the NSAID alone, and after the step of feeding the unit dosage form there occurs a further step of waiting for accelerated blood absorption of the NSAID in the equine.

In one embodiment, the ailment is arthritis, inflammation, fever, laminitis, swelling, founder, joint diseases, joint deterioration, or a combination thereof.

In one embodiment, the unit dosage for, e.g., the cookie, can be conveniently hand-fed to the equine. Hand-feeding a treat, e.g., a cookie, to the equine provides several advantages over current methods of administering NSAIDs, including that specific dosages of the drug can be monitored, and that the animal can be conditioned to readily accept that drug as part of a regular treat schedule.

The invention also provides a method of treating inflammation in an equine comprising administering to an equine in need of such treatment an effective amount of a unit dosage form as described herein, e.g., a bute cookie, wherein the inflammation in the equine is effectively treated. Equines, such as domestic horses, can be effectively trained to readily consume the dosage forms described herein in a manner that is faster and more complete that the manner in which equines eat standard feeds, thereby ensuring regular and effective delivery of NSAIDs to the equine.

### BRIEF DESCRIPTION OF THE DRAWINGS

FIG. 1 illustrates the results of a comparative experiment showing the difference in time of drug administration, test versus control, according to an embodiment of the invention.

FIG. 2 illustrates the results of a bioequivalence test for concentration of phenylbutazone in horse blood post administration, according to an embodiment.

FIG. 3 illustrates the results of a bioequivalence cross-over test for concentration of phenylbutazone in horse blood, test versus control, according to an embodiment.

### DEFINITIONS

The words and phrases presented in this patent application have their ordinary meanings to one of skill in the art unless otherwise indicated. Such ordinary meanings can be obtained by reference to their use in the art and by reference to general and scientific dictionaries, for example, Webster's New World Dictionary, Simon & Schuster, New York, N.Y., 1995, The American Heritage Dictionary of the English Language, Houghton Mifflin, Boston Mass., 1981, and Hawley's Condensed Chemical Dictionary, 14^{th} edition, Wiley Europe, 2002.

The following explanations of certain terms are meant to be illustrative rather than exhaustive. These terms have their ordinary meanings given by usage in the art and in addition include the following explanations.

The term “about” can refer to a variation off 5%, ±10%, ±20%, or ±25% of the value specified. For example, “about 50” percent can in some embodiments carry a variation from 45 to 55 percent. For integer ranges, the term “about” can include one or two integers greater than and/or less than a recited integer. Unless indicated otherwise, the term “about” is intended to include values, e.g., weight percents, proximate to the recited range that are equivalent in terms of the functionality of the individual ingredient, the composition, or the embodiment. In addition, unless indicated otherwise, a recited range (e.g., weight percents) includes each specific value, integer, decimal, or identity within the range.

As used herein, the singular forms “a,” “an,” and “the” include plural reference unless the context clearly dictates otherwise. Thus, for example, a reference to “a formulation” includes a plurality of such formulations, so that a formulation of compound X includes formulations of compound X.

As used herein, the term “and/or” refers to any one of the items, any combination of the items, or all of the items with which this term is associated.

As used herein, the term “additive” refers to any edible element, compound, and/or ingredient that is added either singularly or in combination to enhance at least one of the stability, texture, density, flavor, color and/or shelf life of a cookie.

As used herein, the term “cookie” refers to a small cake, usually flat and often having a sweet flavor. The term “cookie” is synonymous with the term “biscuit” in the United Kingdom. Cookies may be broadly classified according to how they are formed. These classifications include, for example, drop cookies, refrigerator cookies, molded cookies, pressed cookies, bar cookies, sandwich cookies, and fried cookies. The cookie can optionally be baked. Cookies are usually round, however, they can take any form according to their final processing. Other forms of cookies include bars, wafers, and the like.

As used herein, the term “drug” refers to a chemical capable of administration to an organism which modifies or alters the organism's physiology. More preferably, as used herein, the term “drug” refers to any substance intended for use in the treatment or prevention of disease, particularly for humans or equines. Drug includes synthetic and naturally occurring toxins and bioaffecting substances as well as recognized pharmaceuticals, such as those listed in The Merck Index, 14^{th} Ed., Merck Research Laboratories, Whitehouse Station, N.J., 2006, The Physicians Desk Reference, 62^{nd} edition, 2008, pages 101-201, Thomson Healthcare Inc., Montvale, N.J., Goodman and Gilman's The Pharmacological Basis of Therapeutics, 8^{th} Edition (1990), pages 84-1614 and 1655-1715, and The United States Pharmacopeia, The National Formulary, USP XXII NF XVII (1990), the compounds of these references being herein incorporated by reference.

As used herein, the term “effective amount” refers to an amount of bioactive agent (e.g., a drug), an acceptable salt thereof, a derivative thereof, or any combination of those useful to treat or prevent an underlying disorder or disease, or to treat the symptoms associated with the underlying disorder or disease in a host.

As used herein, the term “emulsifier” refers to any compound or agent that aids in forming an emulsion from one or more constituents.

As used herein, the term “equine” refers to any member of the genus Equus, such as a horse, donkey, or zebra, preferably E. Caballus, a domestic horse.

As used herein, the term “flavoring agent” refers to flavored compounds or compositions used in food or food supplement to impart a desired taste and/or aroma.

As used herein, the term “food dye” refers to a substance that is used in the manufacture of foods for purposes of color correction or to produce a pleasant appearance.

As used herein, the term “grain product” refers to a small hard seed or seed-like fruit of any cereal plant, for example, wheat, rice, corn, rye, and the like.

As used herein, the term “mineral” refers to an inorganic nutrient required for a variety of biochemical functions which cannot be synthesized by the body and must be supplied by the diet.

As used herein, the phrase “in one embodiment” refers a particular feature, structure, or characteristic. However, every embodiment may not necessarily include the particular feature, structure, or characteristic. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, the term “non-steroidal anti-inflammatory drug” refers to any non-opioid analgesics that acts like an anti-inflammatory, analgesic, or anti-pyretic agents. Examples include, but are not limited to, diclofenac, etodolac, ketorolac, bromfenac, ibuprofen, fenoprofen, fluriboprofen, ketoprofen, naproxen, suprofen, meclofenamate, mefenamic acid, piroxicam, meloxicam, indomethacin, sulindac, phenylbutazone, firocoxib, oxyphenbutazonetolmetin, and celecoxib.

As used herein and in the appended claims, the term “nutrient” refers to any substance which helps support life, such as a carbohydrate, protein, fat, mineral, or vitamin.

As used herein, the terms “pharmaceutically acceptable salts” or “acceptable salts” refer to derivatives wherein the parent compound is modified by making acid or base salts thereof. Examples of acceptable salts include, but are not limited to, mineral or organic acid salts of basic residues such as amines, alkali or organic salts of acidic residues such as carboxylic acids, and the like. The acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric, and the like, and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Specifically, the acceptable salts can include those salts that naturally occur in vivo in a mammal.

As used herein, the terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “palatable” refers to a substance or material that is agreeable or pleasing to the sense of taste.

As used herein, the term “protein product” refers to a material that is rich in amino acids. Protein products are primarily of animal origin (e.g., meat), but can be of plant origin (e.g., legume).

As used herein, the term “stabilizer” refers to any compound or agent that aids in stabilizing an emulsion from one or more constituents.

As used herein, the terms “treating,” “treat,” or “treatment” includes (i) preventing a pathologic condition (e.g., fever or inflammation) from occurring (e.g., prophylaxis), (ii) inhibiting the pathologic condition (e.g., fever or inflammation) or arresting its development, and (iii) relieving the pathologic condition (e.g., relieving the symptoms associated with fever or inflammation).

As used herein, the term “unit dosage form” refers to an amount of active pharmaceutical ingredient or drug sufficient for achieving a therapeutic effect in a target population.

As used herein, the term “vitamin” refers to an organic nutrient required in small quantities for a variety of biochemical functions which in some cases cannot be synthesized by the body and must be supplied by the diet.

### DETAILED DESCRIPTION OF INVENTION

The present invention provides dosage forms for the delivery of one or more non-steroidal anti-inflammatory drugs to horses or related animals that are palatable, desirable, and easy to administer with little or no mess or waste. The dosage form is a cookie that includes one or more grain products, one or more protein products, one or more flavoring agents, or a combination thereof. The dosage form also includes a therapeutically effective amount of one or more non-steroidal anti-inflammatory drugs for the treatment of joint deterioration, swelling and inflammation, founder, fever, laminitis, or a combination thereof.

It has been surprisingly discovered that a unit dosage form of one or more non-steroidal anti-inflammatory drugs can be conveniently prepared by incorporating one or more non-steroidal anti-inflammatory drugs into a cookie that is specially designed for the taste buds of equines. Equines find the cookies to be delicious and the handlers find the cookies to a convenient and easy drug delivery method.

Each cookie can be made with a specific amount of one or more non-steroidal anti-inflammatory drugs along with any of a variety of other palatable ingredients, such as grain products, protein products, flavoring agents, vitamins, minerals, preservatives, and amino acids. As a result, the cookie can be designed to palatable, healthy, and have a long shelf life. The cookies can be packaged for veterinary and consumer use. When the drug is phenylbutazone, the cookie can be referred to as a “bute cookie.”

### Non-Steroidal Anti-Inflammatory Drugs

Non-steroidal anti-inflammatory drugs (“NSAIDs”) are non-opioid analgesics characterized in that they are nonsteroidal drugs which act as anti-inflammatory, analgesic and anti-pyretic agents. This class of drugs is well known in the art. See, for example, The Pharmacological Basis of Therapeutics, 8th edition, Goodman et al., Chapter 26 (1990). These drugs share certain therapeutic actions and side effects. Within this broad class of drugs are salicylates, for example, aspirin, pyrazolone derivatives, for example, phenylbutazone, onyphenbutazone, antipyrine, aminopyrine, dipyrone and apazone, indomethacin, sulindac, fenamates, for example, mefenamic, meclofenamic, flufenamic, tolfenamic and etofenamice acids, aryl acetic acid and propionic acid compounds, for example, 2-(p-isobutylphenyl)propionic acid (generic name ibuprofen), α-methyl-4-(2-thienylcarbonyl)benzene acetic acid (generic name suprofen), 4,5-diphenyl-2-oxazole propionic acid (generic name oxprozin), rac-6-chloro-alphamethyl-carbazole-2-acetic acid (generic name carprofen), 2-(3-phenyloxyphenyl)-propionic acid, the calcium salt dihydrate thereof (these compounds being referred to generically as fenoprofen and fenoprofen calcium), 2-(6-methoxy-2-naphthyl)propionic acid (generic name naproxen, the generic name of the sodium salt is naproxen sodium), 4-(1,3-dihydro-1-oxo-2H-isoindol-2-yl)-a-methylbenzene acetic acid (generic name indoprofen), 2-(3-benzoylphenyl)propionic acid (generic name ketoprofen), and 2-(2-fluoro-4-biphenylyl)propionic acid (generic name flurbiprofen), and 1-5-(4-methylbenzoyl)-1H-pyrrole-2-acetic acid (generic name tolmetin). Non-steroidal anti-inflammatory drugs also include, for example, sodium 5-(4-chlorobenzoyl)-1,4-dimethyl-1H-pyrrole-2-acetate dihydrate (generically referred to as zomepirac sodium), 4-hydroxy-2-methyl-N-(2-pyridyl-2H-1,2-benzothiazine-3-carboxamide-1,1-dioxide (generic name piroxicam), 2′,4′-difluoro-4-hydroxy-3-biphenylcarboxylic acid (generic name diflunisal), or 1-isopropyl-7-methyl-4-phenyl-2(1H)-quinozolinone (generic name proquazone), phenylacetic acid derivatives, for example, diclofenac, etodolac and nabumetone. All of the non-steroidal anti-inflammatory drugs (“NSAIDs”) are commercially available materials.

Suitable non-steroidal anti-inflammatory drugs (“NSAIDs”) include, for example, diclofenac, etodolac, ketorolac, bromfenac, ibuprofen, fenoprofen, fluriboprofen, ketoprofen, naproxen, suprofen, meclofenamate, mefenamic acid, piroxicam, meloxicam, indomethacin, sulindac, phenylbutazone, firocoxib, oxyphenbutazonetolmetin, celecoxib, or a pharmaceutically acceptable salt, metabolite, or prodrug thereof, or a combination thereof.

Preferably, the one or more non-steroidal anti-inflammatory drug is phenylbutazone. Phenylbutazone is commonly known in the art and is described in U.S. Pat. No. 2,562,830. Phenylbutazone is also known as 4-butyl-diphenyl-3,5-pyrazoidinedione, benzone, butadione, intrabutazone, and by numerous other common names. Phenylbutazone is widely understood to be an effective veterinary anti-inflammatory and analgesic agent in treating inflammation in horses and other animals. Phenylbutazone is an acidic, lipophilic, non-steroidal anti-inflammatory drug, which is extensively metabolized in the horse. The metabolites include, for example, oxyphenbutazone and γ-hydroxyoxyphenbutazone, which account for some 25-30% of administered dose over 24 hours. The plasma half-life of phenylbutazone and termination of its pharmacological action are determined primarily by its rate of hepatic metabolism. Phenylbutazone acts by inhibiting the cyclooxygenase enzyme system, which is responsible for synthesis of prostanoids such as PGE2. Phenylbutazone appears to act on prostaglandin-H synthase and prostacyclin synthase, after conversion by prostaglandin-H synthase to reactive intermediates. Phenylbutazone markedly reduces prostanoid-dependent swelling, edema, erythema, and hypersensitivity to pain in inflamed tissues. There are numerous commercial suppliers of phenylbutazone including, for example, Sigma Corporation (St. Louis, Mo. USA).

Phenylbutazone may also be incorporated in the unit dosage form by using, for example, premixed and flavored phenylbutazone formulations as described in, for example, U.S. Pat. No. 6,022,563, or as provided by suppliers such as Vedco, Inc., (St. Joseph, Mo., U.S.A.). Other suitable sources of phenylbutazone include, for example, apple-flavored phenylbutazone paste (Luitpold Pharmaceuticals, Inc., Shirley, N.Y.), which can also be incorporated into the cookie formulations described herein.

The one or more non-steroidal anti-inflammatory drugs are present in an amount ranging from about 0.1 percent by weight to about 50 percent by weight of the unit dosage form, preferably in an amount ranging from about 1 percent by weight to about 10 percent by weight of the unit dosage form, and most preferably in an amount ranging from about 2 percent by weight to about 6 percent by weight of the unit dosage form.

### Grain Products

A grain product is a small, hard seed or seed like fruit of any cereal plant, for example, barley. Suitable grain products may include for example, amaranth, barley, bran, buckwheat, corn, flax, millet, oats, rice, sorghum, triticale, rice, wheat, and the like. Preferred grain products include, for example, bran, oats, or a combination thereof.

The one or more grain products are present in an amount ranging from about 5 percent by weight to about 45 percent by weight of the unit dosage form, preferably in an amount ranging from about 15 percent by weight to about 35 percent by weight of the unit dosage form, and most preferably in an amount ranging from about 21 percent by weight to about 27 percent by weight of the unit dosage form.

### Protein Products

A protein product is a material that is rich in amino acids. Protein products are primarily of animal origin (e.g., meat), but can be of plant origin (e.g., legume).

Suitable proteins products include, for example, albumin, alfalfa, cheese, egg, milk, peanut, soy, whey, or a combination thereof. Preferred protein products include, for example, alfalfa, whey, or a combination thereof.

The one or more protein product are present in an amount ranging from about 0.1 percent by weight to about 50 percent by weight of the unit dosage form, preferably in an amount ranging from about 1 percent by weight to about 25 percent by weight of the unit dosage form, and most preferably in an amount ranging from about 3 percent by weight to about 14 percent by weight of the unit dosage form.

### Flavoring Agents

A flavoring agent is a flavored compound or compositions used in food or a food supplement to impart a desired taste and/or aroma. The flavoring agent may be one or more sweeteners and flavor additives that make the cookie palatable to horses. The flavoring agent may be any type of compatible sweetener, either from a natural material, an artificially produced sweetener, or a combination thereof. A natural flavoring agent may be, for example, an essential oil, oleoresin, essence or extractive, protein hydrolysate, distillate, or any product of roasting, heating or enzymolysis, which contains the flavoring constituents derived from a spice, fruit or fruit juice, vegetable or vegetable juice, edible yeast, herb, bark, bud, root, leaf or any other edible portions of a plant, meat, seafood, poultry, eggs, dairy products, or fermentation products thereof, whose primary function in food is flavoring rather than nutritional. An artificial flavoring agent is a chemically synthesized compound that are used to flavor food items but do not meet the specifications listed above.

Suitable artificial sweeteners may include, for example, acesulfame-K (available from Nutrinova, Frankfurt, Germany), aspartame (available from Monsanto Corporation, St. Louis, Mo., USA), saccharine (available from Monsanto Corporation, St. Louis, Mo., USA), or sucralose (available from McNeil Nutritionals LLC, Fort Washington, Pa., USA). Suitable natural sweeteners may include molasses, sucrose, glucose, fructose, lactose, and dextrose.

Suitable flavorants may include, for example, synthetic flavor oils and flavoring aromatics and/or oils, oleoresins, extracts derived from plants, leaves, flowers, fruits, and the like, and combinations thereof. Suitable flavor oils may include, for example, spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Suitable flavoring agents also may include, for example, artificial, natural and synthetic fruit flavors such as vanilla, citrus oils (e.g., lemon, orange, lime, and grapefruit), and fruit essences (e.g., apple, pear, peach, grape, strawberry, raspberry, cherry, plum, pineapple, and apricot), and the like, and combinations thereof. The flavoring agents may be used, for example, in liquid or solid form and may be used individually or in admixture. Suitable other flavorants may include, for example, certain aldehydes and esters, e.g., cinnamyl acetate, cinnamaldehyde, citral diethylacetal, dihydrocarvyl acetate, eugenyl formate, p-methylamisol, and the like, and combinations thereof.

Preferred flavoring agents may include, for example, anise, apple fiber, banana, carrot, cherry, citric acid, cinnamon, fenugreek, molasses, orange, salt, raspberry, strawberry, vanillin, naturally expressed citrus or spice oils, or a combination thereof. More preferably, a combination of anise, apple fiber, fenugreek, and molasses is used.

The one or more flavoring agents are present in an amount ranging from about 0.1 percent by weight to about 50 percent by weight of the unit dosage form, preferably in an amount ranging from about 4 percent by weight to about 30 percent by weight of the unit dosage form, and most preferably in an amount ranging from about 8 percent by weight to about 20 percent by weight of the unit dosage form.

### Additives

The cookies described herein may also include various additional additives to enhance their stability, texture, density, flavor, color and/or shelf life. For example, the cookies may include various food dyes, emulsifiers, fragrances, humectant, minerals, nutrients, preservatives, salt, seasonings, stabilizers, vitamins, and the like, or combinations thereof.

Food dyes may be incorporated to improve the acceptance of the cookie by the horse. Suitable food dyes may be both of natural and of synthetic origin. Suitable natural food dyes may include, for example, plant dyes such as carotenoids, flavonoids and anthocyans, animal dyes such as cochineal, and inorganic pigments like titanium dioxide, iron oxide pigments, and iron hydroxide pigments. Food dyes may also include, for example, products of enzymatic browning like polyphenols and products of nonenzymatic browning like melanoidines as well as products of heating such as sugar coloring and caramel. Synthetic food dyes may include, for example, azo, triphenylmethane, indigoid, xanthene and quinoline compounds. Synthetic food dyes may include, for example, FD&C Blue No. 1, FD&C Blue No. 2, FD&C Green No. 3, FD&C Red No. 3, FD&C Red No. 40, FD&C Yellow No. 5 and FD&C Yellow No. 6.

Suitable emulsifiers may include, for example, monoglycerides and diglycerides, polysorbates, sodium lauryl sulfate, sucrose esters and lecithin.

Suitable fragrances may include, for example, fragrances that are described more fully in S. Arctander, Perfume Flavors and Chemicals, Vols. I and II, Arctander, Montclair, N.J. (1969) and The Merck Index, 14^{th} Edition, Merck & Co., Inc. Rahway, N.J. (2006), both references being incorporated herein by reference.

Suitable humectants may include, for example, sucrose, fructose, lactose, dextrose, maltose, galactose, sorbose, mannose, maple syrup, corn syrups, invert syrups, high fructose corn syrups, honey, molasses, glycerol, mannitol, maltitol, xylitol, sorbitol, propylene glycol, hydrogenated glucose syrups, sugar esters, dextrins, hydrogenated starch hydrolysates, other starch hydrolysis products, and combinations thereof.

Suitable minerals may include, for example, calcium, phosphorus, magnesium, sodium, potassium, chloride, zinc, iron, copper manganese, selenium, iodine, chromium, and combinations thereof.

Suitable nutrients may include, for example, proteins, amino acids, fats, fatty acids, and combinations thereof.

Suitable food preservatives may include, for example, antimicrobials (e.g., sulfur dioxide or sulfites, propionates, benzoates, and nitrites) that inhibit growth of various microorganisms, antioxidants (e.g., butylated hydroxytoluene, tert-butylhydroquinone, and propyl gallate) that slow air-induced oxidation of fats and lipids to prevent rancidity, and preservatives (e.g., phenolase, citric acid and ascorbic acid) that block the natural ripening and enzymatic changes in foods such as fresh produce. Suitable preservatives may also include, for example, sodium benzoate, potassium sorbate, propionic acid, sodium propionate, sodium dehydroacetate and other organic acids and their salts, butyl paraoxybenzoate, isobutyl paraoxybenzoate, propyl paraoxybenzoate and like organic acid esters, sodium sulfite, sodium hyposulfite, potassium pyrosulfite, sodium pyrosulfite, sulfur dioxide and like inorganic salts, hinokitiol, peptin extract and like plant extracts or plant decomposed products, and milt protein extract, E-polylysine glycine, chitosan and like proteins and their decomposed products.

Suitable seasonings may include, for example, garlic, cloves, onion, chili pepper, black pepper, sweet basil, bay leaf, marjoram, parsley, sage, rosemary, thyme, and the like, and combinations thereof.

Suitable stabilizers may include, for example, various gums including xanthan, guar, karaya, locust bean, methylcellulose, carboxymethylcellulose, carageenan, agar, and pectin.

Suitable vitamins may include, for example, vitamin A, vitamin D, Vitamin E, Vitamin K, Vitamin C, thiamin, riboflavin, pantothenate/pantothenic acid, niacin, pyridoxine, folate/folic acid, biotin, vitamin B₁₂, choline, and combinations thereof.

### Dosage Levels

A typical moderate phenylbutazone dosage level for a 1000-pound horse (average size) is 1-2 grams, or 5-10 milliliters of a solution, per administration. Three daily dosages are usually administered to assure a somewhat constant level blood concentration. The plasma half-life of phenylbutazone is 5-6 hours in horses. The maximum oral dose recommended by manufacturers is 2 to 4 grams per 1000 pounds of body weight (4 to 9 mg/kg) per day. Manufacturers recommend that the dose be divided equally and given every 8 hours for maximum results, although most horse owners give phenylbutazone every 12 to 24 hours for convenience, usually giving 1 to 2 grams in the morning and at night. In some horses, however, phenylbutazone may irritate the digestive tract. As such, a cookie as described herein, for example, a bute cookie, may be administered with omeprazole, cimetidine, and/or sucralfate to alleviate the irritation, or the cookie may include omeprazole, cimetidine, and/or sucralfate as an ingredient.

The following Examples are intended to illustrate the above invention and should not be construed as to narrow its scope. One skilled in the art will readily recognize that the Examples suggest other ways in which the invention could be practiced. It should be understood that numerous variations and modifications may be made while remaining within the scope of the invention.

### EXAMPLES

Unless otherwise indicated, all numbers expressing quantities of ingredients, properties such as molecular weight, preparation conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

As would be readily understood by one skilled in the art, any of the cookies described below can be prepared using NSAIDs other then phenylbutazone to obtain similar results. Specific examples of the invention include cookies as described below in the Examples where the phenylbutazone ingredient has been replaced with another NSAID, such as firocoxib, or another NSAID recited herein, or a combination thereof.

### Example 1

### Preparation of Cookies without Phenylbutazone

The dry and liquid ingredients listed in Table 1 were mixed in separate bowls and combined. The combination was folded together and placed by spoonful on parchment papered cookie sheets to dry to afford cookies weighing 14-21 grams each. The first sheet afforded 20 cookies at 14 grams/cookie. The second sheet afford afforded 11 cookies at 21 grams/cookie. The cookies were dried with a hair dryer at low setting for 5 minutes. The cookies were air-dried for 24 hours and were found to be moist. The cookies were then air-dried for an additional 24 hours and found to be sufficiently dry (i.e., semi-soft).

The semi-soft 14 gram cookies were eagerly consumed by three horses and sufficiently consumed by two horses.

TABLE 1

| Ingredient | Amount | |
|---|---|---|
| Oat bran | 130 | grams |
| Flax meal | 20 | grams |
| All-purpose flour | 170 | grams |
| Brewer's yeast | 7.5 | grams |
| Cornstarch | 7.5 | grams |
| Molasses | 80 | grams |
| Applesauce | 252.5 | grams |

### Example 2

### Preparation of Cookies without Phenylbutazone

The dry and liquid ingredients listed in Table 2 were mixed in separate bowls and combined. The combination was folded together but it was found not to bind well.

TABLE 2

| Ingredient | Amount | |
|---|---|---|
| Whole wheat flour | 80 | grams |
| Corn meal | 10 | grams |
| Apple fiber | 3.7 | grams |
| Salt | 15 | grams |
| Brewer's yeast | 3.7 | grams |
| Molasses | 15 | grams |
| Corn oil | 10.5 | grams |

### Example 3

### Preparation of Cookies without Phenylbutazone

The corn oil and molasses listed in Table 3 were mixed and added to a mixture of the dry ingredients. Water was added and the dough was kneaded by hand. The dough was rolled into 21 gram size balls and placed on cookie sheet to afford 7 cookies (6 cookies at 21 grams/cookie and 1 cookie at 24 grams/cookie). The cookies had an attractive smell and texture. The cookies were air-dried for 9 hours to afford five cookies weighing 20 grams, one cookie weighing 18 grams, and one cookie weighing 22 grams. The cookies were stored in a plastic bag for one week and found to have good texture.

TABLE 3

| Ingredient | Amount | |
|---|---|---|
| Whole wheat flour | 80 | grams |
| Water | 71 | grams |
| Corn meal | 10 | grams |
| Apple fiber | 10 | grams |
| Guar gum | 3.7 | grams |
| Alfalfa meal | 10 | grams |
| (calcium carbonate + silica) | | |
| Molasses | 21 | grams |
| Dried Molasses Meal | 7 | grams |
| Corn oil | 21 | grams |
| Fenugreek (ground) | 3.5 | grams |

### Example 4

### Preparation of Cookies with Phenylbutazone

The dry ingredients listed in Table 4 were mixed and the corn oil was then added. To this was added a mixture of phenylbutazone in water. The combination was thoroughly mixed and molasses was added. The dough was rolled and formed into nine cookies (21 grams/cookie). The cookies were air-dried overnight on a waxed paper cookies sheet to afford cookies of excellent smell and texture.

The cookies were placed in individual plastic bags. Two cookies were stored at room temperature, five cookies were stored in the refrigerator, and two cookies were stored in a plastic bag. The two cookies that stored in a plastic bag for two days were then fed to a lame horse, which showed improvement in ambulation within the hour.

TABLE 4

| Ingredient | Amount | |
|---|---|---|
| Oat flour | 30 | grams |
| Water | 71 | grams |
| Corn meal | 10 | grams |
| Apple fiber | 10 | grams |
| Guar gum | 3.7 | grams |
| Alfalfa meal | 29 | grams |
| (calcium carbonate + silica) | | |
| Molasses | 21 | grams |
| Dried Molasses Meal | 7 | grams |
| Corn oil | 21 | grams |
| Fenugreek (ground) | 7 | grams |
| Phenylbutazone tablets | 12 tablets at 1 gram/tablet | |
| Citric acid | 7 | grams |

### Example 5

### Preparation of Cookies with Phenylbutazone

The ingredients listed in Table 5 were mixed as follows. Mixing containers were wiped with rubbing alcohol to prevent sticking. The alfalfa and phenylbutazone tablets were mixed together in water. To this was added corn oil and molasses and stirred. The dry ingredients were combined in a separate container, mixed, and were combined with the mixture of the wet ingredients. The dough was kneaded by hand and cut to form ten 7.75 gram cookies, which were put on a waxed paper baking sheet. The cookies were baked at 200° F. for 20 minutes, cooled to room temperature, and air-dried for one day. The cookies were fed to four horses. Three of the horses readily consumed the cookies and one showed indifference.

TABLE 5

| Ingredient | Amount | |
|---|---|---|
| Oat flour | 35 | grams |
| Water | 85 | grams |
| Corn meal | 11 | grams |
| Apple fiber | 10 | grams |
| Guar gum | 5 | grams |
| Alfalfa tablets | 43 tablets (35 grams) | |
| Molasses | 17.5 | grams |
| Dried Molasses Meal | 10 | grams |
| Corn oil | 15 | grams |
| Fenugreek (ground) | 9 | grams |
| Phenylbutazone tablets | 14 tablets at 1 gram/tablet | |
| Citric acid | 10 | grams |

### Example 6

### Preparation of Cookies without Phenylbutazone

The ingredients listed in Table 6 were mixed as follows. The mixing containers were wiped with rubbing alcohol to prevent sticking. The alfalfa tablets were dissolved in the water. The dry ingredients were combined in a separate container, mixed, and combined with the alfalfa dissolved in the water. The dough was kneaded by hand and cut to form cookies, which were air-dried. The cookies were reluctantly consumed by the horses.

TABLE 6

| Ingredient | Amount | |
|---|---|---|
| Bran | 100 | grams |
| Water | 340 | grams |
| Apple fiber | 18 | grams |
| Alfalfa tablets | 66 tablets (50 grams) | |
| Dried Molasses Meal | 20 | grams |
| Fenugreek (ground) | 10 | grams |
| Citric acid | 2 | grams |

### Example 7

### Preparation of Cookies without Phenylbutazone

The ingredients listed in Table 7 were mixed as follows. The mixing containers were wiped with rubbing alcohol to prevent sticking. The alfalfa tablets were dissolved in the water. The dry ingredients were combined in a separate container, mixed, and combined with the alfalfa dissolved in the water. The dough was kneaded by hand and cut to form ten 36.5 gram cookies, which were baked at 200° F. for 5 minutes, and air-dried to afford 26 gram cookies. Two of the horses readily consumed the cookies and one horse partially comsumed the cookie.

TABLE 7

| Ingredient | Amount | |
|---|---|---|
| Bran | 50 | grams |
| Water | 340 | grams |
| Apple fiber | 18 | grams |
| Alfalfa tablets | 66 tablets (50 grams) | |
| Dried Molasses Meal | 15 | grams |
| Fenugreek (ground) | 15 | grams |
| Citric acid | 2 | grams |

### Example 8

### Preparation of Cookies without Phenylbutazone

The ingredients listed in Table 8 were mixed as follows. The mixing containers were wiped with rubbing alcohol to prevent sticking. The alfalfa tablets were dissolved in the water. The dry ingredients were combined in a separate container, mixed, and combined with the alfalfa dissolved in the water. The dough was kneaded by hand and cut to form ten cookies, which were baked at 200° F. for 5 minutes, and air-dried to afford ten 31.5 gram cookies. Four the horses readily consumed the cookies.

TABLE 8

| Ingredient | Amount | |
|---|---|---|
| Bran | 25 | grams |
| Oat Flour | 100 | grams |
| Water | 340 | grams |
| Apple fiber | 23 | grams |
| Alfalfa tablets | 33 tablets (25 grams) | |
| Dried Molasses Meal | 10 | grams |
| Fenugreek (ground) | 15 | grams |
| Citric acid | 1 | gram |
| Guar gum | 1 | gram |

| | |
|---|---|
| Anise | A few drops |

### Example 9

### Preparation of Cookies with Phenylbutazone

The ingredients listed in Table 9 were mixed as follows. The mixing containers were wiped with rubbing alcohol to prevent sticking. The alfalfa tablets were dissolved in the water. The dry ingredients were combined in a separate container, mixed, and combined with the alfalfa dissolved in the water. The dough was kneaded by hand and cut to form ten cookies, which were baked at 200° F. for 5 minutes, and air-dried for 2 days to afford ten 31.5 gram cookies. Three of the horses readily consumed the cookies and wanted more while one horse showed indifference.

TABLE 9

| Ingredient | Amount | |
|---|---|---|
| Bran | 20 | grams |
| Oat Flour | 95 | grams |
| Water | 340 | grams |
| Apple fiber | 23 | grams |
| Phenylbutazone powder with apple | 23 | grams |
| flavoring | | |
| Alfalfa tablets | 20 | grams |
| Dried Molasses Meal | 10 | grams |
| Fenugreek (ground) | 15 | grams |
| Citric acid | 1 | gram |
| Guar gum | 1 | gram |

| | |
|---|---|
| Anise | A few drops |

### Example 10

### Preparation of Cookies with Phenylbutazone

The ingredients listed in Table 10 were mixed as follows. The mixing containers were wiped with rubbing alcohol to prevent sticking. The alfalfa tablets were dissolved in the water. The dry ingredients were combined in a separate container, mixed, and combined with the alfalfa dissolved in the water. The dough was kneaded by hand and cut to form cookies, which were air-dried for 1 day. Three of three horses readily consumed the cookies and showed increased mobility after one hour.

TABLE 10

| Ingredient | Amount | |
|---|---|---|
| Bran | 20 | grams |
| Oat Flour | 95 | grams |
| Water | 340 | grams |
| Apple fiber | 23 | grams |
| Phenylbutazone powder | 13 | grams |
| Alfalfa tablets | 20 | grams |
| Dried Molasses Meal | 10 | grams |
| Fenugreek (ground) | 15 | grams |
| Citric acid | 1 | gram |
| Guar gum | 1 | gram |

| | |
|---|---|
| Anise | A few drops |

### Example 11

### Taste Test of Cookies with Phenylbutazone against Pellets coated with Phenylbutazone

The ingredients listed in Table 11 were mixed as follows. The mixing containers were wiped with rubbing alcohol to prevent sticking. The alfalfa tablets were dissolved in the water. All of the dry ingredients, except phenylbutazone, were combined in a separate container, mixed, and combined with the alfalfa dissolved in the water. The dough was kneaded by hand and cut to form 11 cookies. To each cookie was added 1 gram of phenylbutazone powder. Each cookie was kneaded by hand and then baked at 200° F. for 6 minutes. The cookies were cooled to room temperature and stored in plastic bags at room temperature for 11 days.

Two feed pans were prepared as follows: 350 grams SAFE CHOICE® pellets (Nutrena brand, Cargill Inc.) coated with flavored phenylbutazone powder was added to one side of each feed pan. Two cookies as prepared above were added to the other side of each feed pan.

The first feed pan was offered to an old geriatric and arthritic horse. The horse took one bite of the pellets and then promptly consumed all of the two cookies. In one hour, the horse exhibited improved movement and expression.

The second feed pan was offered to another horse. The horse first ate both cookies, nibbled at the pellets, and then abandoned the pellets. The horse later exhibited improved movement and expression.

TABLE 11

| Ingredient | Amount | |
|---|---|---|
| Oat Bran | 20 | grams |
| Oat Flour | 95 | grams |
| Water | 170 | grams |
| Apple fiber | 20 | grams |
| Phenylbutazone powder | 11 | grams |
| Alfalfa tablets | 20 | grams |
| Dried Molasses Meal | 10 | grams |
| Fenugreek (ground) | 15 | grams |
| Citric acid | 1 | gram |
| Guar gum | 1 | gram |
| Anise | 1.2 | grams |
| Anise Seeds | 0.5 | grams |

### Example 12

### Palatability and Time Efficiency Study

This is a single dose palatability and time efficiency study of an orally, hand fed “Bute Cookie”, prepared according to Example 11, administered to healthy horses.

Objective: Assessment of phenylbutazone “Bute Cookie” in the matter of palatability and time efficiency. The purpose is to demonstrate that the phenylbutazone cookie would be readily accepted and consumed in a more efficient and timely manner than the control product (“bute powder”; Vedco, Inc., St. Joseph, Mo.) mixed with Safe Choice® pelleted horse feed.

Study Animals: Twenty-eight (28) domestic breed horses (17 geldings, one stallion and 10 mares, ages 2-27) were randomly selected.

Treatment: The horses received the Test Product (Cookie, Group A), and the Control Product (Powder, Group B). Notations were made regarding how much of and how fast the products were consumed.

Dosage Form: Test product (Cookie) contained one-gram of phenylbutazone per cookie; control product (Powder) contained 1 gram of phenylbutazone per 1 heaping gram scoop, top dressed on Safe Choice® pelleted horse feed.

Route of Administration: The Test Product (Cookie) was hand delivered to each horse, orally accepted and consumed. Control product (Powder) was top dressed on ½ scoop (½ gallon) of Nutrena Safe Choice® pelleted horse feed, placed in a feed pan and offered to the horse in the typical manner. The Powder was orally consumed with the pelleted feed. In the cases of finicky horses, the product was removed after 30 minutes if not consumed and the uneaten portion was determined. Horses were not fasted prior to testing in order to accurately duplicate actual feeding and administration scenarios.

Observations: Prior to testing, each horse was observed to be in good health and condition.

Statistical Analysis: Notations of the observations were tabled.

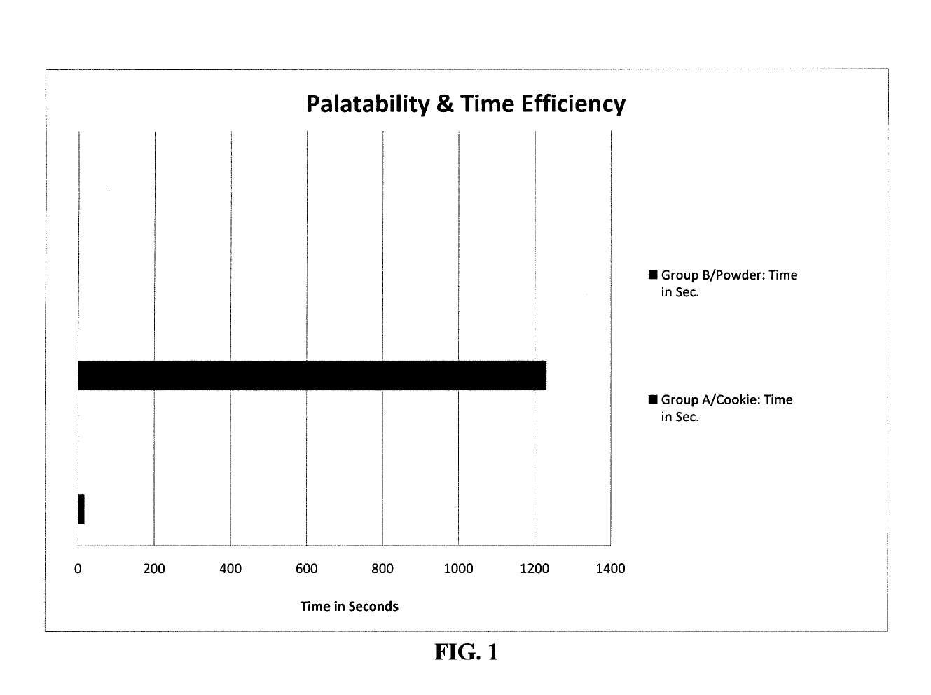

Results: The results are shown in Table 12 and FIG. 1.

Adverse Reactions No adverse reactions to test or control products were observed during this test.

Conclusion: The Test Product (Cookie) was found to be accepted and consumed significantly faster and more completely than the Control Product (Powder). This efficiency vastly improves the ability of horse owner's certainty of consumption and precise dosage.

TABLE 12
Palatability & Time Efficiency Study

| Group A | Test Product: Bute Cookie | | | | Group B | Control Product: Powdered Bute | | | |
|---|---|---|---|---|---|---|---|---|---|
| Horse | Age | Gender | Time/sec. | Amt | Horse | Age | Gender | Time/min | Amt |
| 1 | 26 | G | 18.2 | 100 | 1 | 26 | G | 18 | 100 |
| 2 | 9 | G | 13.3 | 100 | 2 | 9 | G | 30 | 0 |
| 3 | 15 | S | 14.2 | 100 | 3 | 15 | S | 14 | 100 |
| 4 | 15 | S | 14.5 | 100 | 4 | 15 | S | 15 | 100 |
| 5 | 2 | G | 14.6 | 90 | 5 | 2 | G | 16 | 100 |
| 6 | 15 | M | 12.8 | 100 | 6 | 15 | M | 12 | 100 |
| 7 | 14 | G | 14.1 | 100 | 7 | 14 | G | 17 | 100 |
| 8 | 26 | G | 19.7 | 100 | 8 | 26 | G | 30 | 50 |
| 9 | 15 | M | 14.6 | 100 | 9 | 15 | M | 14 | 100 |
| 10 | 2 | G | 16.5 | 100 | 10 | 2 | G | 30 | 50 |
| 11 | 8 | M | 16.6 | 100 | 11 | 8 | M | 11 | 100 |
| 12 | 18 | G | 17.2 | 90 | 12 | 18 | G | 30 | 75 |
| 13 | 18 | M | 18.8 | 100 | 13 | 18 | M | 20 | 100 |
| 14 | 15 | G | 15.4 | 100 | 14 | 15 | G | 30 | 75 |
| 15 | 26 | G | 18.9 | 100 | 15 | 26 | G | 30 | 60 |
| 16 | 15 | M | 14.6 | 100 | 16 | 15 | M | 30 | 0 |
| 17 | 2 | G | 13.2 | 100 | 17 | 2 | G | 18 | 100 |
| 18 | 6 | G | 17.2 | 100 | 18 | 6 | G | 11 | 100 |
| 19 | 9 | G | 15.4 | 100 | 19 | 9 | G | 12 | 100 |
| 20 | 12 | G | 18.3 | 100 | 20 | 12 | G | 13 | 100 |
| 21 | 4 | M | 18.6 | 80 | 21 | 4 | M | 15 | 100 |
| 22 | 27 | G | 19.4 | 100 | 22 | 27 | G | 16 | 100 |
| 23 | 16 | M | 13.1 | 100 | 23 | 16 | M | 30 | 75 |
| 24 | 3 | G | 14.6 | 100 | 24 | 3 | G | 30 | 50 |
| 25 | 6 | G | 16.5 | 100 | 25 | 6 | G | 30 | 75 |
| 26 | 5 | M | 15.8 | 100 | 26 | 5 | M | 16 | 100 |
| 27 | 4 | M | 16.3 | 100 | 27 | 4 | M | 17 | 100 |
| 28 | 6 | G | 19.2 | 100 | 28 | 6 | G | 14 | 100 |
| | | TTL | 451.6 | 2760 | | | TTL | 569 | 2210 |
| | | AVG | 16.1 sec. | 98.6 | | | AVG | 20.3 min. | 78.92 |

### Example 13

### Bio-Equivalence Blood Test of “Bute Cookie” and “Bute Powder”

This is a single dose, single draw bio-equivalence study with orally dispensed phenylbutazone in healthy horses.

Objective: Assessment of Test Product, a phenylbutazone “Bute Cookie”, to Control product, a phenylbutazone Powder, to demonstrate that the Cookie would be fully consumed and offer equivalent blood level concentration as the Control product. Assessment via in vivo bioequivalence of “Cookie” (Test product) in horses compared to phenylbutazone Powder (Control product, U.S. Pat. No. 6,022,563).

Study Animals: Eight (8) domestic breed horses (six geldings, one stallion and one mare, ages 9-26) were randomly assigned to either of two treatment groups.

Treatment: The study was a one period design. Group A received the Test Product (Cookie), prepared according to Example 11. Group B received the Control Product (Powder).

Dosage Form: Control product (Powder; Vedco, Inc., St. Joseph, Mo.) contains 1 gram of phenylbutazone per 1 heaping gram scoop, 2 scoops each horse, mixed in ½ gallon of Nutrena Safe Choice® pellet horse feed (a typical feed for horses, usually fed twice daily). The Test product (Bute Cookie) contained one-gram of phenylbutazone per cookie; 2 cookies each horse.

Route of Administration: Test Product (Cookie) was hand delivered to each horse and orally accepted and consumed. (Approximate time to consume was 15-30 seconds.) Control product was top dressed on ½ scoop (½ gallon) of Nutrena Safe Choice® pelleted horse feed. Powder was orally consumed with the pelleted feed (approximate time to consume was 12-16 minutes). Horses were not fasted prior to testing in order to more accurately duplicate realistic feeding and administration scenarios. It is not realistic to fast horses prior to phenylbutazone administration. However, the amount of feed in the digestive tract and stomach, especially hay, can affect and delay blood assimilation and possibly decrease peak plasma concentrations. Other factors that can affect blood assimilation readings include age (younger horses are known to metabolize phenylbutazone faster), hormone levels of mares and stallions, and the general disposition of the horse can (see, e.g., Piperno et al., “Plasma & Urine Levels of Phenylbutazone in the Horse,” J. Am. Vet. Med. Assoc., 1968, 153(2), 1958).

Dosage: 2 Grams of phenylbutazone per horse (each horse weighing approximately 1000 lbs.; 8.8 mg/kg body wt.).

Observations: Prior to testing, each horse was observed to be in good health and condition. None of the test horses had received any medications for two weeks (14 days) prior to testing.

Analytical Methods: One blood sample was collected at 4.25 hours after dose administration (5 cc of blood was collected from each horse; extracted via a 20 gauge 1½″ needle into a 13×100 mm 7 mL tube). Blood specimens were refrigerated at 40 degrees Fahrenheit, packaged in styrofoam and shipped to the Texas Veterinary Medical Diagnostic Lab in College Station, Tex. Plasma was harvested and assayed for phenylbutazone concentration.

Statistical Analysis: Phenylbutazone concentration levels in plasma were extracted from blood samples and measured at 4.25 hours after dosage (approximate peak concentration time).

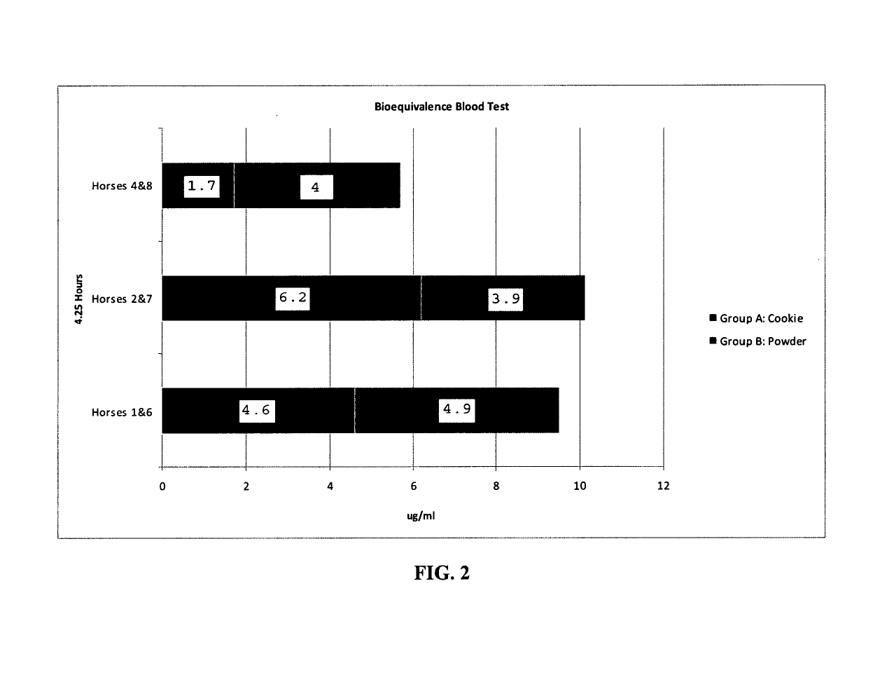

Results: Results from horses three and five were discarded due to a zero/negative blood result. The results are shown in Table 13 and FIG. 2.

Adverse Reactions: No adverse reactions to test or control products were observed during this test.

Conclusion: The test product (Cookie) was found to be therapeutically bioequivalent to the control product (Powder).

TABLE 13

| | Group A | | Group B |
|---|---|---|---|
| Horse | Cookie μg/mL | Horse | Powder μg/mL |
| 1 | 4.6 | 6 | 4.9 |
| 2 | 6.2 | 7 | 3.9 |
| 4 | 1.7 | 8 | 4.0 |

### Example 14

### Bio-Equivalence Blood Test of “Bute Cookie” and “Bute Powder”

This is a single dose, four draw bio-equivalence crossover study with orally dispensed phenylbutazone in healthy horses.

Objective: Assessment of Test Product, phenylbutazone “Bute Cookie”, to reference product, phenylbutazone Powder to demonstrate that the generic phenylbutazone “cookie” would be consumed and offer equivalent blood level concentration as the Control Product. Assessment via in vivo bioequivalence of “Cookie” (Test product) in horses compared to phenylbutazone powder (Control product, U.S. Pat. No. 6,022,563). The bute cookie test product was prepared according to Example 11.

Study Animals: Three (3) domestic breed horses (two geldings, ages 2 and 26, and one (1) mare, age 15).

Treatment: The study was a two period design. Group A: Phase I received the Control Product (Powder). Group B: Phase II received the Test Product (Cookie).

Dosage Form: Control product (Powder) contains 1 gram of phenylbutazone per 1 heaping gram scoop, 2 scoops each horse, mixed in ½ gallon of Nutrena Safe Choice® pellet horse feed (a typical feed for horses, usually fed twice daily). The Test product (Cookie) contained one-gram of phenylbutazone per cookie; 2 cookies each horse.

Route of Administration: Control product was top-dressed on ½ scoop (½ gallon) of Nutrena Safe Choice® pelleted horse feed. Powder was orally presented and consumed with the pelleted feed. Horses were allowed free choice access of the top-dressed feed, which was left in front of horses for a period of 30 minutes. In that time only one horse (Horse 1) completely finished the top-dressed feed [16 minutes to consume]. Horse 2 consumed 75% of the top-dressed feed and horse 3 consumed less than 50% of the top-dressed feed.

Horses were not fasted prior to testing in order to more accurately duplicate true life scenarios. It is not realistic to fast horses prior to phenylbutazone administration. However, the amount of feed in the digestive tract and stomach, especially hay, can affect and delay blood assimilation and possibly decrease peak plasma concentrations. Also, the age of the horse can affect the amounts found in plasma, because younger horses have higher metabolism and will process the drug more quickly. Mares and stallions will have a different level of hormones that can affect readings as well as the mental state of the horse. Nervous horses will also cause the drug to be metabolized more quickly (see, e.g., Piperno et al., “Plasma & Urine Levels of Phenylbutazone in the Horse,” J. Am. Vet. Med. Assoc. 1968, 153(2), 1958).

Test Product (Bute Cookie) was hand delivered to each horse and was freely and orally accepted and consumed. The approximate time to consume the cookies was a notable 12-20 seconds per cookie.

Horse 1: 19.4 seconds+18.9 seconds=37.4 seconds

Horse 2: 13.1 seconds+14.6 seconds=27.7 seconds

Horse 3: 14.6 seconds+13.2 seconds=27.8 seconds

Average=15.6 seconds per cookie; 31.3 seconds for two cookies.

Dosage: 2 Grams (8.8 mg/kg body wt.) of phenylbutazone per horse (each horse weighing approximately 1000 lbs.) for each phase of the test.

Observations: Prior to testing, each horse was observed to be in good health and condition. None of the test horses had received any medications for two weeks (14 days) prior to testing, nor had they received drugs during the standard wash-out period.

Analytical Methods: Blood samples were collected at 2.0; 4.0; 6.0; and 8.0; hours after dose administration (5 cc of blood was collected from each horse; extracted from the jugular vein via 20 gauge 1½″ needle into a 13×100 mm 7 mL tube). Blood specimens were refrigerated at 40 degrees Fahrenheit, packaged in styrofoam and shipped to the Texas Veterinary Medical Diagnostic Lab in College Station, Tex. Plasma was harvested and assayed for phenylbutazone concentration.

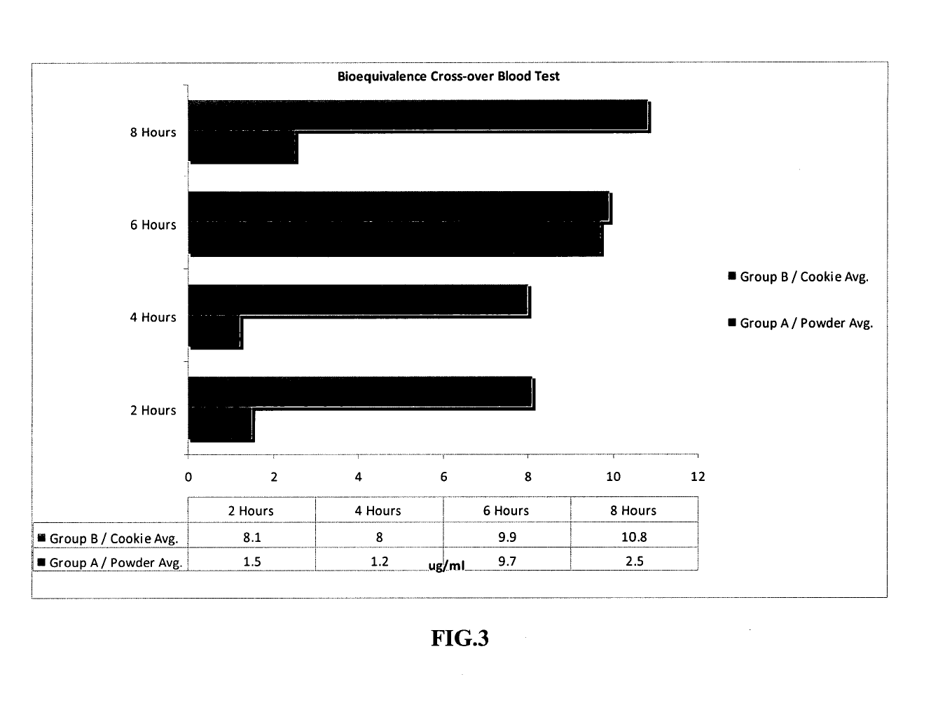

Statistical Analysis: Phenylbutazone concentration levels in plasma were assayed by the Texas Veterinary Medical Diagnostic Lab in College Station, Tex. The samples taken were positive for phenylbutazone activity in the serum and were tabled (FIG. 3).

Results: The Test Product (Cookie) blood levels proved to have therapeutic and bio-equivalent levels to the commonly used Control product (Powder). The results are shown in FIG. 3.

Adverse Reactions: No adverse reactions to test or control products were observed during this test.

Conclusion: The Test product (Cookie) was found to be present in all horses tested; notably, it surpassed the level of concentration of activity found in the Control Product blood samples and appeared to have greater concentration in a shorter period of time in addition to being present in blood for a longer duration. This can be attributed to the fact that dosage may be more accurate and consumption is faster when delivered in the Cookie form. The horses took considerably longer to ingest the Powder form and often left significant amounts of feed uneaten, thus leaving the actual dosage uncertain. Various amounts of uneaten powder had sifted to the bottom of the feeder and was left uneaten.

As such, the delivery methods of the Bute Cookie fulfills the pharmacological efficiency and the advantages as described herein. The time in which the horses consumed the Cookie compared to the time it took to consume the Control Product (Powder) was considerably faster. Further, the dosage via the Cookie is more reliable.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicant reserves the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims.

1. A unit dosage form for the delivery of one or more non-steroidal anti-inflammatory drugs to an equine comprising:
a cookie comprising one or more grain products, one or more protein products, and optionally one or more flavoring agents, or a combination thereof and
a therapeutically effective amount of one or more non-steroidal anti-inflammatory drugs for the treatment of an equine ailment, wherein the ailment is joint deterioration, swelling and inflammation, founder, fever, laminitis, or a combination thereof.

2. The unit dosage form of claim 1, wherein the one or more non-steroidal anti-inflammatory drugs comprise diclofenac, etodolac, ketorolac, bromfenac, ibuprofen, fenoprofen, fluriboprofen, ketoprofen, naproxen, suprofen, meclofenamate, mefenamic acid, piroxicam, meloxicam, indomethacin, sulindac, phenylbutazone, firocoxib, oxyphenbutazonetolmetin, celecoxib, or a pharmaceutically acceptable salt thereof, or a combination thereof.

3. The unit dosage form of claim 2, wherein the one or more non-steroidal anti-inflammatory drugs is phenylbutazone or firocoxib.

4. (canceled)

5. The unit dosage form of claim 1, wherein non-steroidal anti-inflammatory drug is phenylbutazone and the ingredients of the cookie in combination with the phenylbutazone are present in an amount effective to accelerate blood absorption of phenylbutazone into equine blood subsequent to oral consumption of the unit dosage form, wherein acceleration of blood absorption is determined with respect to administration of phenylbutazone alone.

6. The unit dosage form of claim 1, wherein the one or more grain products comprise amaranth, barley, bran, buckwheat, corn, flax, millet, oats, rice, sorghum, triticale, rice, wheat, or a combination thereof.

7. (canceled)

8. The unit dosage form of claim 1, wherein the one or more protein products comprise albumin, alfalfa, cheese, egg, milk, peanut, soy, whey, or a combination thereof.

9. (canceled)

10. The unit dosage form of claim 1, wherein the one or more flavoring agents comprises anise, apple fiber, banana, carrot, cherry, citric acid, cinnamon, fenugreek, molasses, orange, salt, raspberry, strawberry, vanillin, naturally expressed citrus or spice oils, or a combination thereof.

11. (canceled)

12. The unit dosage form of claim 1, wherein the one or more non-steroidal anti-inflammatory drugs are present in an amount ranging from about 2 percent by weight to about 6 percent by weight of the unit dosage form.

13. A method of administering one or more non-steroidal anti-inflammatory drugs to an equine comprising administering a cookie to an equine, wherein the cookie comprises:
one or more grain products, one or more protein products, and optionally one or more flavoring agents, or a combination thereof, and a therapeutically effective amount of one or more non-steroidal anti-inflammatory drugs for the treatment of an equine ailment;
wherein the ailment is joint deterioration, swelling and inflammation, founder, fever, laminitis, or a combination thereof.

14. The method of claim 13, wherein the one or more non-steroidal anti-inflammatory drugs comprise diclofenac, etodolac, ketorolac, bromfenac, ibuprofen, fenoprofen, fluriboprofen, ketoprofen, naproxen, suprofen, meclofenamate, mefenamic acid, piroxicam, meloxicam, indomethacin, sulindac, phenylbutazone, firocoxib, oxyphenbutazonetolmetin, celecoxib, or a pharmaceutically acceptable salt, metabolite, or prodrug thereof, or a combination thereof.

15. (canceled)

16. The method of claim 13, wherein the one or more grain products comprise amaranth, barley, bran, buckwheat, corn, flax, millet, oats, rice, sorghum, triticale, rice, wheat, or a combination thereof.

17. (canceled)

18. The method of claim 13, wherein the one or more flavoring agents comprises anise, apple fiber, banana, carrot, cherry, citric acid, cinnamon, fenugreek, molasses, orange, salt, raspberry, strawberry, vanillin, naturally expressed citrus or spice oils, or a combination thereof.

19. The method of claim 18, wherein the one or more flavoring agents comprises anise, apple fiber, citric acid, fenugreek, molasses, or a combination thereof.

20. The method of claim 13, wherein the one or more non-steroidal anti-inflammatory drug is present in an amount ranging from about 2 percent by weight to about 6 percent by weight of the unit dosage form.

21. The method of claim 13, wherein the cookie is hand-fed to the horse.

22. A unit dosage form for the delivery of phenylbutazone to a horse comprising:
a cookie comprising one or more grain products, one or more protein products, and optionally one or more flavoring agents, or a combination thereof and
a therapeutically effective amount of phenylbutazone for the treatment of an equine ailment, wherein the ailment is joint deterioration, swelling and inflammation, founder, fever, laminitis, or a combination thereof, wherein the phenylbutazone is present in an amount ranging from about 2 percent by weight to about 6 percent of the unit dosage form.

23. A method of administering phenylbutazone to an equine comprising feeding the unit dosage form of claim 1 to the equine;
wherein the phenylbutazone is dispersed substantially homogeneously in the unit dosage form in an amount ranging from about 2 percent by weight to about 6 percent by weight of the unit dosage form.

24. The method of claim 23, wherein the equine is a horse.

25. The method of claim 23, wherein the ailment is arthritis, inflammation, fever, laminitis, swelling, founder, joint diseases, joint deterioration, or a combination thereof.

26. A method of treating inflammation in a horse comprising administering to a horse in need of such treatment an effective amount of the unit dosage form of claim 1, wherein the inflammation in the horse is effectively treated.
